(19) United States
(12) Patent Application Publication
(10) Pub. No.: US 2024/0409485 A1
(43) Pub. Date: Dec. 12, 2024

(54) PROCESS FOR PREPARING CRESOL FROM DITOLYLETHER

(71) Applicant: LANXESS DEUTSCHLAND GMBH, COLOGNE (DE)

(72) Inventors: Paul SPRENGER, Cologne (DE); Thomas KAESE, Leverkusen (DE); Olaf HALLE, Cologne (DE); Hans-Jürgen QUELLA, Leverkusen (DE); Eileen GILLESSEN, Leverkusen (DE); Jürgen KARTHAUS, Solingen (DE); Ulrich NOTHEIS, Dormagen (DE)

(73) Assignee: LANXESS DEUTSCHLAND GMBH, COLOGNE (DE)

(21) Appl. No.: 18/682,955

(22) PCT Filed: Aug. 11, 2022
(86) PCT No.: PCT/EP2022/072585

§ 371 Date: Feb. 12, 2024

(30) Foreign Application Priority Data
Aug. 13, 2021 (EP) 21191354.6

(51) Int. Cl. C07C 37/52; B01J 23/30; B01J 29/18; B01J 29/22; B01J 29/40; B01J 29/44; B01J 29/46

(52) U.S. Cl. CPC C07C 37/52; B01J 23/30; B01J 29/18; B01J 29/22; B01J 29/40; B01J 29/44; B01J 29/46

(57) ABSTRACT

The present invention relates to a novel process for preparing cresol from ditolyl ether.

The present invention relates to novel processes for producing cresol from ditolyl ether.

### PRIOR ART

It is known that the C—O bonds of aromatic ethers may be cleaved by hydrolysis or hydrogenolysis in the presence of radioactive thorium oxide for example (DE-A 2604474) but this does not enable industrial applications.

The use of nickel/nickel compounds on various substrates, such as carbon or aluminum oxide/silicon oxide, in the hydrogenolysis of C—O bonds in diaryl ethers in the presence of relatively large amounts of sterically demanding strong bases, for example NaOtBu or potassium hexamethyldisilazide, is also known, see Gao et al. Applied Chem. Int. Ed. 2016, 55, 1474-1478. Disadvantages of this include the long reaction time, the high proportion of the transition metal nickel and also the necessary use of sterically demanding strong bases which require costly and complex separation.

### OBJECT OF THE PRESENT INVENTION

It is accordingly an object of the present invention to provide an improved process for producing cresol from ditolyl ether that allows simple production of cresol in high yield and with high selectivity.

### ACHIEVEMENT OF THE OBJECT

It has now been found that, surprisingly, the object can be achieved when ditolyl ether and water are reacted in the presence of a catalyst containing at least 2 of the following oxides selected from aluminum oxide, silicon oxide, titanium oxide, zirconium oxide and/or tungsten oxide.

### SUBJECT MATTER OF THE INVENTION

The present invention provides a process for producing cresol from ditolyl ether and water in the presence of catalysts containing at least 85% by weight of at least 2 of the following oxides selected from aluminum oxide, silicon oxide, titanium oxide, zirconium oxide and/or tungsten oxide

It is preferable when a catalyst consisting of at least 2 of the following oxides titanium oxide, zirconium oxide and tungsten oxide and/or a catalyst from the group of zeolites which contains at least 85% by weight of at least 2 of the following oxides selected from aluminum oxide, silicon oxide, titanium oxide, zirconium oxide and tungsten oxide is employed.

Cresol in the context of the invention comprises o-cresol, m-cresol and p-cresol as individual compounds and as a mixture. Mixtures may comprise any desired ratios of o-, m-, and p-cresol.

Ditolyl ether in the context of the invention comprises 2,2′-, 2,3′-, 2,4′-, 3,3′-, 3,4′- and/or 4,4′-ditolyl ether as individual compounds and as a mixture of 2 or more of these individual aforementioned compounds.

Preference is given to the use of a mixture of 2,2′-, 2,3′-, 2,4′-, 3,3′-, 3,4′- and 4,4′-ditolyl ethers as obtained for example in the alkaline hydrolysis of chlorotoluenes (see H. Fiege, Cresols and Xylenols, Ullmann's Encyclopedia of Industrial Chemistry, page 427, vol. 10, Wiley-VCH Verlag GmbH & Co. KGaA, Weinheim, 2012.) Water in the context of the invention preferably comprises demineralized water.

Catalyst in the context of the invention is to be understood as meaning that a catalyst which contains at least 85% by weight, preferably 90-100% by weight, of at least 2 of the following oxides selected from aluminum oxide, silicon oxide, titanium oxide, zirconium oxide and tungsten oxide is employed.

In a preferred embodiment the catalyst consists of at least 2 of the following oxides titanium oxide, zirconium oxide and tungsten oxide and/or of the group of zeolites containing at least 85% by weight of at least 2 of the following oxides selected from aluminum oxide, silicon oxide, titanium oxide, zirconium oxide and tungsten oxide.

In a preferred embodiment the catalyst from the group of zeolites contains silicon oxide, aluminum oxide, titanium oxide and/or zirconium oxide.

It is preferable when the catalyst preferably contains silicon oxide and aluminum oxide and the molar ratio of silicon to aluminum is preferably 3:1 to 300:1, particularly preferably 5:1 to 250:1, very particularly preferably 30:1 to 90:1.

In a particularly preferred embodiment the catalyst has the structure of a zeolite. Zeolite in the context of the invention is preferably to be understood as meaning a microporous material whose structure is characterized by a framework of vertex-linked tetrahedra. Each tetrahedron generally consists of four oxygen atoms that surround a cation. Preferred cations for these tetrahedra are preferably selected from aluminum and silicon. The zeolite structure preferably consists of oxygen-aluminum tetrahedra and oxygen-silicon tetrahedra.

As is well known every zeolite cell structure is characterized by a 3-letter code by the structure commission of the International Zeolite Association (IZA).

The zeolites are particularly preferably mixtures of aluminum oxide and silicon oxide of the framework types:
LTA=Linde type A (e.g.: Zeolite A),
MFI=Mobil Five (e.g.: ZSM-5, Zeolite Socony Mobil-5),
MOR=Mordenite,
BEA=Beta Zeolite and/or
FAU=Faujasite (e.g.: Zeolite X,Y).

Very particularly preferred framework types are MII and MOR.

The zeolite may be used as a powder or as a shaped body. For shaping as a shaped body the zeolite is preferably admixed and processed with 5-70% by weight of aluminum- or silicon-based binder material and various inorganic and organic auxiliaries, for example nitric acid, citric acid, acetic acid, methylcellulose, glycerol, polyethylene glycol, sugar, and/or starch. Processed in the present case comprises the steps of shaping, for example by pressing, and heating at temperatures in the range from 350° C. to 900° C.

The process according to the invention preferably employs, from the group of MFI zeolites, commercially available Z SM-5 zeolites (Zeolite Socony Mobil-5) and MFI zeolites, for example from Clariant Produkte (Deutschland) GrnbH.

The ZSM-5 and MFI host structure is preferably characterized by ten-membered rings which form a three-dimensional and intersecting pore system. Furthermore, as is typical for “high-silica” zeolites and pentasil zeolites, there is a large number of five-membered rings (in addition to a small number of four-, six-, seven- and eight-membered rings).

The general chemical composition of the group of zeolites is as follows for Al oxide and Si oxide for example:

$${{M}^{n +}}_{x / n} [ {{( {AlO}_{2} )}^{-}}_{x} {( {SiO}_{2} )}_{y} ] \cdot z {H}_{2} O$$

Mⁿ⁺ is a cation, preferably H⁺, Na⁺ and/or NH₄⁺, wherein n denotes the charge of the cation and is preferably 1 oder 2. The cations Mn⁺ are generally not part of the structure-defining aluminum-oxygen-tetrahedra and silicon-oxygen-tetrahedra rather, but are disposed in the zeolite channels or cavities for charge balancing. In the preferred embodiment H⁺ is employed as the cation, as may be characterized in the notation H-ZSM-5 and H-MFI. By chemical derivatization, for example by ion exchange reactions, the type of cation can be reversibly altered.

The molar ratio of oxygen-silicon tetrahedra to oxygen-aluminum tetrahedra (SI/Al ratio for short) is referred to as the modulus and expressed by the quotient y/x. In the preferred form y/x is 3-300, particularly preferably 30-90. The quotient may be characterized in the notation H-MFI-y/x, i.e. for example H-MFI-90.

Employable catalysts for the process according to the invention include commercially available mixed oxide catalysts, preferably in the form of spheres or extrudates. Such catalysts are for example mixtures of ZrO₂/WO₃ and ZrO₂/SiO₂ from Saint-Gobain Ceramic Materials GmbH and Al₂O₃/SiO₂ from Shell Catalysts & Technologies Leuna GmbH. Employable mixtures of Al₂O₃/SiO₂ further include commercially available zeolites, preferably in the form of spheres or extrudates. Examples of commercially available zeolites are extrudates of MFI zeolites, MOR zeolites, BEA zeolites, such as for example from Clariant Produkte (Deutschland) GmbH, and FAU zeolites, such as for example from Zeolyst International.

The process according to the invention is preferably performed at temperatures of 250° C. to 450° C., preferably 270° C. to 400° C., particularly preferably 300° C. to 370° C.

The process according to the invention is preferably performed at a pressure of 0.5 bar to 300 bar, particularly preferably 0.9 bar to 50 bar and very particularly preferably 1 bar to 10 bar.

The process according to the invention is preferably performed in reactors.

Employable reactors include all vessels that allow addition of gases and where the catalyst is in the form of a fixed bed, for example fixed bed reactors, and those where the catalyst is moved by the reactant feed or a stirring apparatus, for example entrained flow reactors, fluidized bed reactors or batch reactors. The reactants may be added in gaseous or liquid form. In the preferred embodiment the reactants are added as gases to a fixed bed reactor with a fixed catalyst bed. The process according to the invention may be performed continuously or discontinuously.

The total amount of catalyst, based on ditolyl ether (DTE), may be selected as desired for continuously operated reactors or discontinuously operated reactors. For continuously operated reactors said amount is preferably 0.01-1000 g DTE/(g catalyst×h), particularly preferably 0.01-100 g DTE/(g catalyst×h). In the case of discontinuously operated reactors said amount is preferably 0.1-10000 g DTE/g cat.

The molar ratio of ditolyl ether to water is preferably 10:1 to 1:40, particularly preferably 1:1 to 1:20, very particularly preferably 1:5 to 1:15.

In addition to ditolyl ether and water a further gas, preferably an inert gas, such as nitrogen or argon, may be added. It is preferable to add as many standard liters Lₙ of nitrogen or argon as are required to achieve a proportion based on the total volume of 0-95% by volume, particularly preferably 0-40% by volume.

Nitrogen in the context of the invention preferably has a purity greater than 99% by volume.

In this embodiment the process for producing cresol from ditolyl ether and water in the presence of a catalyst may also be referred to as a hydrolytic cleavage of the ditolyl ether.

In this preferred embodiment of the invention it is preferable to perform the process in a continuous mode as follows:

The catalyst is initially charged in a fixed bed reactor and heated to 300° C. to 370° C. A mixture of ditolyl ether and water preheated to at least 250° C. in a molar ratio in a range from 1:5 to 1:15, admixed with not more than 40% by volume of nitrogen, is added at 0.1 g to 5 g of ditolyl ether per g of catalyst per hour. The reaction product formed is collected since this contains cresol formed in high yield.

In a further preferred embodiment of the invention the production of cresol from ditolyl ether is effected by hydrogenolysis by reaction in the presence of water, hydrogen and a catalyst which additionally contains at least one metal from the group of platinum metals (Ru, Rh, Pd, Os, Ir, Pt).

An advantage of the hydrogenolytic production of cresol from ditolyl ether is the simultaneous generation of toluene.

In this preferred embodiment of the invention reference is made to the abovementioned definitions and embodiments in respect of cresol, ditolyl ether and water.

Hydrogen in the context of the invention preferably has a purity greater than 99% by volume.

Catalyst in the context of the invention in respect of the hydrogenolysis is to be understood as meaning that a catalyst which contains at least 85% by weight, preferably 90-99.9% by weight, of at least 2 of the following oxides selected from aluminum oxide, silicon oxide, titanium oxide, zirconium oxide and tungsten oxide and in addition at least one element from the group of platinum metals is employed.

In a preferred embodiment, the catalyst contains silicon oxide and aluminum oxide or zirconium oxide and tungsten oxide and in addition at least one element from the group of platinum metals.

It is preferable when the molar ratio of silicon oxide to aluminum oxide is preferably 3:1 to 300:1, particularly preferably 5:1 to 250:1, very particularly preferably 30:1 to 90:1.

In a particularly preferred embodiment the catalyst has the structure of a zeolite. In respect of the definition of zeolite in the context of the invention reference is made to the foregoing.

The zeolites are particularly preferably mixtures of aluminum oxide and silicon oxide of the framework types:
LTA Linde type A (e.g.: Zeolite A),
MFI=Mobil Five (e.g.: ZSM-5, Zeolite Socony Mobil-5),
MOR=Mordenite,
BEA=Beta Zeolite and/or
FAU=Faujasite (e.g. Zeolite X,Y).

Very particularly preferred framework types are MFI and MOR.

For the elements from the group of platinum metals a proportion of 0.1-10% by weight, based on the total amount of the catalyst, is preferred. A proportion of 0.5-5% by weight is particularly preferred for the elements from the group of platinum metals.

It is further preferable when the metal from the group of platinum metals is platinum and/or rhodium.

Platinum metal-containing catalysts may be produced by commonly used production processes for noble metal-containing catalysts. This may be done for example by contacting a metal salt solution of the noble metal with the catalyst support by impregnation or spraying. The impregnation may be performed as an immersion-impregnation where the volume of aqueous solution is greater than the liquid absorption volume of the catalyst support to be coated or as a dry impregnation where the volume of aqueous solution is not more than the liquid absorption volume of the catalyst support to be coated. The same applies to the spray-application of noble metal solutions to the catalyst support. It is further possible to perform an ion exchange process to add noble metals to the catalysts, for example by repeatedly adding a diluted metal salt solution of the noble metal over a catalyst support. Impregnation, spraying or ion exchange is followed by drying of the noble metal-containing catalyst, preferably in an air stream of elevated temperature, preferably at 60° C. to 200° C. for 0.5 h to 10 h. It is optionally also possible to perform a calcination at elevated temperature, preferably at 200° C. to 1000° C. for 10 min to 24 h.

The process according to the invention is particularly preferably performed at temperatures of 250° C. to 450° C., preferably 270° C. to 400° C., particularly preferably 300° C. to 370° C.

The process according to the invention is preferably performed at a pressure of 0.5 bar to 300 bar, particularly preferably 0.9 bar to 50 bar and very particularly preferably 1 bar to 10 bar.

The process according to the invention is preferably performed in reactors.

Employable reactors include all vessels that allow addition of gases and where the catalyst is in the form of a fixed bed, for example fixed bed reactors, and those where the catalyst is moved by the reactant feed or a stirring apparatus, for example entrained flow reactors, fluidized bed reactors or batch reactors. The reactants may be added in gaseous or liquid form. In the preferred embodiment the reactants are added as gases to a fixed bed reactor with a fixed catalyst bed. The process according to the invention may be performed continuously or discontinuously.

The total amount of catalyst, based on ditolyl ether (DTE), may be selected as desired for continuously operated reactors or discontinuously operated reactors. For continuously operated reactors said amount is preferably 0.01-1000 g DTE/(g catalyst×h), particularly preferably 0.01-100 g DTE/(g catalyst×h). In the case of discontinuously operated reactors said amount is preferably 0.1-10000 g DTE/g cat.

The molar ratio of ditolyl ether to water is preferably 10:1 to 1:40, particularly preferably 1:1 to 1:20, very particularly preferably 1:5 to 1:15.

The molar ratio of ditolyl ether to hydrogen is preferably 10:1 to 1:100, particularly preferably 1:1 to 1:50, very particularly preferably 1:5 to 1:40.

In addition to ditolyl ether and water and hydrogen a further gas, preferably an inert gas, such as nitrogen or argon, may be added. It is preferable to add as many standard liters Lₙ of nitrogen or argon as are required to achieve a proportion based on the total volume of 0-95% by volume, particularly preferably 0-40% by volume.

Employable reactors include all vessels that allow addition of gases and where the catalyst is in the form of a fixed bed, for example fixed bed reactors, and those where the catalyst is moved by the reactant feed or a stirring apparatus, for example entrained flow reactors, fluidized bed reactors or batch reactors. The reactants may be added in gaseous or liquid form. In the preferred embodiment the reactants are added as gases to a fixed bed reactor with a fixed catalyst bed.

In this preferred embodiment of the invention as hydrogenolysis the following performance of the process in continuous mode is preferred:

The catalyst is initially charged in a fixed bed reactor and heated to 300° C. to 370° C. A mixture, preheated to at least 250° C., of ditolyl ether, water and hydrogen in a molar ratio of ditolyl ether to water in a range from 1:5 to 1:15 and in a molar ratio of ditolyl ether to hydrogen in a range from 1:5 to 1:40, admixed with not more than 40% by volume of nitrogen, is added at 0.1 g to 5 g of ditolyl ether per g of catalyst per hour. The reaction product formed is collected since this contains cresol formed in high yield.

The process according to the invention is elucidated on the basis of the examples that follow, without being restricted thereto.

### EXAMPLES

The experiments specified below were performed in a steel tube having a perforated bottom plate as a reactor. Information on the type and manufacturer of the catalyst for each experiment is reported in table 1 and table 2. A gaseous mixture of ditolyl ether, water and nitrogen and/or hydrogen in the ratios reported in tables 3 and 4 was introduced into the reactor. After the reaction the product mixture was cooled to room temperature and acetone was added to obtain a single-phase mixture that was analyzed by gas chromatography with flame ionization. The results are listed in tables 3 and 4.

TABLE 1
Employed catalysts (CAT)

| | | | Si:Al | Trade name of respective CAT |
|---|---|---|---|---|
| No. | CAT | Composition | ratio | before application of noble metal |
| 1 (C) | Al₂O₃ | Al₂O₃ | — | Alumina Spheres 1.8/210, Sasol |
| | | | | Germany GmbH |
| 2 (I) | ZrO₂, 18% by | ZrO₂/ WO₃ | — | SZ 61143, Saint-Gobain Ceramic |
| | wt. WO₃ | | | Materials GmbH |
| 3 (I) | H-MFI-30 | SiO₂/Al₂O₃ | 30:1 | Clariant Produkte (Deutschland) |
| | | | | GmbH |
| 4 (I) | H-MFI-55 | SiO₂/Al₂O₃ | 55:1 | Süd-Chemie AG |
| 5 (I) | H-MFI-60 | SiO₂/Al₂O₃ | 60:1 | Süd-Chemie AG |
| 6 (I) | H-MFI-90 | SiO₂/Al₂O₃ | 90:1 | HCZP 90E, Clariant Produkte |
| | | | | (Deutschland) GmbH |
| 7 (I) | H-MOR-40 | SiO₂/Al₂O₃ | 40:1 | HCZM 40E, Clariant Produkte |
| | | | | (Deutschland) GmbH |
| 8 (I) | H-MFI-30, | SiO₂/Al₂O₃ | 30:1 | Clariant Produkte (Deutschland) |
| | 0.5% by wt. | | | GmbH |
| | platinum | | | |
| 9 (I) | H-MFI-55, | SiO₂/Al₂O₃ | 55:1 | Süd-Chemie AG |
| | 0.5% by wt. | | | |
| | platinum | | | |
| 10 (I) | H-MFI-90, | SiO₂/Al₂O₃ | 90:1 | HCZP 90E, Clariant Produkte |
| | 0.5% by wt. | | | (Deutschland) GmbH |
| | platinum | | | |
| 11 (I) | H-MFI-240, | SiO₂/Al₂O₃ | 240:1 | Clariant Produkte (Deutschland) |
| | 0.5% by wt. | | | GmbH |
| | platinum | | | |
| 12 (I) | H-MFI-55, | SiO₂/Al₂O₃ | 55:1 | Süd-Chemie AG |
| | 1% by wt. | | | |
| | platinum | | | |
| 13 (I) | H-MOR-40, | SiO₂/Al₂O₃ | 40:1 | HCZM 40E, Clariant Produkte |
| | 1% by weight | | | (Deutschland) GmbH |
| | platinum | | | |
| 14 (I) | H-MFI-90, | SiO₂/Al₂O₃ | 90:1 | HCZP 90E, Clariant Produkte |
| | 1% by wt. | | | (Deutschland) GmbH |
| | rhodium | | | |
| 15 (I) | H-MFI-60, | SiO₂/Al₂O₃ | 60:1 | Süd-Chemie AG |
| | 1% by wt. | | | |
| | platinum, | | | |
| | 0.2% by wt. | | | |
| | nickel | | | |
| 16 (C) | Al₂O₃/SiO₂ with | SiO₂/Al₂O₃ | 1:5 | based on example 6 for diphenyl |
| | 20% by wt. | | | ether in Gao et al. Applied Chem. |
| | nickel | | | Int. Ed. 2016, 55, 1474-1478, |
| | | | | without addition of bases |
| 17 (C) | Norit RX 1.5 | C | — | Norit RX 1.5 Extra, Cabot Norit |
| | Extra | | | Nederland B.V. |
| | activated carbon, | | | |
| | 0.1% by wt. | | | |
| | platinum | | | |

MFI = SiO₂/Al₂O₃ zeolite;
MOR = SiO₂/Al₂O₃ zeolite with mordenite structure
I = inventive,
C = comparative example

The noble metal in the noble metal-containing catalysts (see examples 8 (I) to 15 (I)) was—as explained below—applied by dry impregnation/ion exchange. Amounts and properties are apparent from table 2.

For the dry impregnation the noble metal source specified in table 2, dissolved in demineralized water, was added to a carrier. The amount of demineralized water corresponds to 98% of the absorption capacity of the respective carrier (see table 2). Once the solution was fully absorbed the impregnated carrier was dried at 120° C. for 1 h in a hot air stream and—with the exception of example 16 (C)—calcined at temperatures of 300° C.-500° C. for 12-16 hours in a static oven. In the case of example 16 (C) dry impregnation and drying were repeated three times to allow the entire amount of noble metal solution to be applied.

For the ion exchange the noble metal doping solution was added to a support in a glass tube with a glass frit bottom and the solution was recirculated over the support for 24 h. During this time the carrier was always covered with liquid.

TABLE 2
Amounts and properties of the noble metal-containing catalysts

| | Support before precious | | | | | |
|---|---|---|---|---|---|---|
| | metal doping | | | | | |
| | | Water | Absorption | Doping with precious metal | | |
| | | content/ | capacity/ | | Volume of | |
| | | % by | % by | | solution/ | |
| No. | Mass/g | wt. | wt. | Type | mL | Noble metal source |
| 8 (I) | 51.5 | 3.4 | 43.2 | Dry | 22.2 | 1 g H₂PtCl₆ solution, |
| | | | | impregnation | | 25% by wt. Pt |
| 9 (I) | 51.7 | 3.8 | 40.5 | Dry | 20.9 | 1 g H₂PtCl₆ solution, |
| | | | | impregnation | | 25% by wt. Pt |
| 10 (I) | 101.7 | 2.2 | 42.5 | Ion | 150.0 | 0.9 g |
| | | | | exchange | | [Pt(NH₃)₄]Cl₂ × H₂O |
| 11 (I) | 51.4 | 3.3 | 51.5 | Dry | 25.6 | 1 g H₂PtCl₆ solution, |
| | | | | impregnation | | 25% by wt. Pt |
| 12 (I) | 102.9 | 3.8 | 40.5 | Ion | 150.0 | 1.8 g |
| | | | | exchange | | [Pt(NH₃)₄]Cl₂ × H₂O |
| 13 (I) | 106.4 | 7.0 | 38.2 | Dry | 40.7 | 1 g H₂PtCl₆ solution, |
| | | | | impregnation | | 25% by wt. Pt |
| 14 (I) | 50.6 | 2.2 | 42.5 | Dry | 21.1 | 5 g Rh(NO₃)₃ |
| | | | | impregnation | | solution, |
| | | | | | | 10% by wt. Rh |
| 15 (I) | 51.9 | 4.7 | 46.0 | Dry | 22.7 | 2 g H₂PtCl₆ solution, |
| | | | | impregnation | | 25% by wt. Pt + 0.5 g |
| | | | | | | Ni(NO₃)₂ × 6H₂O |
| 16 (C) | 240.0 | 0.8 | 44.0 | Dry | 320.0 | 291 g |
| | | | | impregnation | | Ni(NO₃)₂ × 6H₂O |
| 17 (C) | 72.2 | 3.2 | 92.8 | Dry | 67.0 | 0.3 g H₂PtCl₆ |
| | | | | impregnation | | solution, |
| | | | | | | 25% by wt. Pt |

TABLE 3
Measured results for hydrolytic cleavage of ditolyl ether at a temperature
of 315° C., a pressure of 1 bar and a catalyst volume of 68
mL. The flows were as follows: 15.2 g/h of ditol ether and 13.8
g/h of water with a proportion of 20% by volume of nitrogen.

| | | DTE | Cresol | Cresol |
|---|---|---|---|---|
| | | conversion/ | selectivity/ | yield/ |
| No. | Catalyst | % | % | % |
| 1 (C) | Al₂O₃ | 1.3 | 51.9 | 0.7 |
| 2 (I) | ZrO₂, 18% by | 23.1 | 54.0 | 12.5 |
| | wt. WO₃ | | | |
| 3 (I) | H-MFI-30 | 13.4 | 74.2 | 9.9 |
| 4 (I) | H-MFI-55 | 12.8 | 68.2 | 8.7 |
| 5 (I) | H-MFI-60 | 14.3 | 70.5 | 10.1 |
| 6 (I) | H-MFI-90 | 7.4 | 84.6 | 6.2 |
| 7 (I) | H-MOR-40 | 15.5 | 46.4 | 7.2 |

C = comparative,
I = inventive

Experiments 2-7 performed according to the inventive process with water showed a high conversion, a high selectivity and a high yield compared to the comparative example, example 1 (C).

Table 4: Measured results for hydrogenolytic cleavage of ditolyl ether at a temperature of 315′C, a pressure of 1 bar and a catalyst volume of 68 mL. The flows were as follows: 15.2 g/h of ditolyl ether and 13.8 g/h of water with a proportion of 2000 by volume of hydrogen.

TABLE 4
Measured results for hydrogenolytic cleavage of ditolyl ether at a
temperature of 315° C., a pressure of 1 bar and a catalyst volume
of 68 mL. The flows were as follows: 15.2 g/h of ditolyl ether and
13.8 g/h of water with a proportion of 20% by volume of hydrogen.

| | | | | | Cresol + |
|---|---|---|---|---|---|
| | | DTE | Cresol | Toluene | toluene |
| | | conversion/ | selectivity/ | selectivity/ | yield/ |
| No. | Catalyst | % | % | % | % |
| 8 (I) | H-MFI-30, | 93.3 | 21.9 | 41.7 | 59.4 |
| | 0.5% by wt. platinum | | | | |
| 9 (I) | H-MFI-55, | 95.3 | 22.3 | 32.2 | 51.9 |
| | 0.5% by wt. platinum | | | | |
| 10 (I) | H-MFI-90, | 86.9 | 22.1 | 40.1 | 54.1 |
| | 0.5% by wt. platinum | | | | |
| 11 (I) | H-MFI-240, | 98.1 | 20.7 | 37.3 | 56.9 |
| | 0.5% by wt. platinum | | | | |
| 12 (I) | H-MFI-55, | 85.4 | 32.4 | 43.4 | 64.8 |
| | 1% by wt. platinum | | | | |
| 13 (I) | H-MOR-40, | 94.2 | 17.7 | 35.1 | 49.7 |
| | 1% by wt. platinum | | | | |
| 14 (I) | H-MFI-90, | 36.3 | 35.1 | 39.4 | 27.1 |
| | 1% by wt. rhodium | | | | |
| 15 (I) | H-MFI-60, | 96.5 | 16.7 | 32.2 | 47.3 |
| | 1% by wt. platinum, | | | | |
| | 0.2% by wt. nickel | | | | |
| 16 (C) ¹⁾ | Al₂O₃/SiO₂ with | 1.8 | 16.3 | 5.1 | 0.4 |
| | 20% by wt. nickel | | | | |
| 17 (C) | Norit RX 1.5 Extra | 4.4 | 32.1 | 42.0 | 3.2 |
| | activated carbon, | | | | |
| | 0.1% by wt. platinum | | | | |

C = comparative experiments,
I = inventive
¹⁾ analogously to Gao et al. Applied Chem. Int. Ed. 2016, 55, 1474-1478.

It was found that the experiments 8 (I)-13 (I) and 15 (I) performed according to the inventive process for platinum-containing catalysts, and in the case of example 14 (I) for rhodium-containing catalysts, achieved a high conversion based on ditolyl ether and a high selectivity and yield based on cresol and toluene relative to the comparative examples 16 (C) and 17 (C).

Comparative example 16 (C) also shows that the prior art use of nickel without addition of a strong base results in an extremely low conversion and a markedly lower selectivity and yield than the inventive catalysts 8 (I)-15 (I) containing at least one element of the platinum group.

1. A process for producing cresol from ditolyl ether and water in the presence of a catalyst, wherein the catalyst consisting of at least 2 of the following oxides titanium oxide, zirconium oxide and tungsten oxide and/or a catalyst from the group of zeolites which contains at least 85% by weight of at least 2 of the following oxides selected from aluminum oxide, silicon oxide, titanium oxide, zirconium oxide and tungsten oxide is employed.

2. The process as claimed in claim 1, wherein the catalyst from the group of zeolites contains silicon oxide, aluminum oxide, titanium oxide and/or zirconium oxide.

3. The process as claimed in claim 1, wherein the catalyst from the group of zeolites contains silicon oxide and aluminum oxide and the molar ratio of silicon to aluminum is 3:1 to 300:1.

4. The process as claimed in claim 1, wherein the catalysts employed are zeolites of the types LTA, MFI, MOR, BEA, FAU.

5. The process as claimed in claim 1, wherein the catalyst additionally contains at least one element from the group of platinum metals and the reaction is performed in the presence of hydrogen.

6. The process as claimed in claim 1, wherein the proportion of the elements from the group of platinum metals is 0.1-10% by weight, based on the total amount of the catalyst.

7. The process according to claim 5, wherein the metal from the group of platinum metals is platinum and/or rhodium.

8. The process according to claim 1, wherein the process is performed at temperatures of 250° C. to 450° C.

9. The process according to claim 14, wherein the process is performed at a pressure of 0.5 bar to 300 bar.

10. The process as claimed in claim 1, wherein the catalyst from the group of zeolites contains silicon oxide and aluminum oxide and the molar ratio of silicon to aluminum is 5:1 to 250:1.

11. The process as claimed in claim 1, wherein the catalyst from the group of zeolites contains silicon oxide and aluminum oxide and the molar ratio of silicon to aluminum is 30:1 to 90:1.

12. The process as claimed in claim 4, wherein the catalysts employed are zeolites of the types MFI or MOR.

13. The process as claimed in claim 1, wherein the proportion of the elements from the group of platinum metals is 0.5-5% by weight, based on the total amount of the catalyst.

14. The process according to claim 1, wherein the process is performed at temperatures of 270° C. to 400° C.

15. The process according to claim 1, wherein the process is performed at temperatures of 300° C. to 370° C.

16. The process according to claim 1, wherein the process is performed at a pressure of 0.9 bar to 50 bar.

17. The process according to claim 1, wherein the process is performed at a pressure of 1 bar to 10 bar.
